SEQUENCE LISTING (84 sequences)

SEQ ID NO: 1 - RNA, 22 nt, Homo sapiens
gcugacuccuaguccagggcuc

SEQ ID NO: 2 - RNA, 21 nt, Homo sapiens
uuucccuuuccauccuggcag

SEQ ID NO: 3 - RNA, 23 nt, Homo sapiens
cuacccucggucugcuuaccaca

SEQ ID NO: 4 - RNA, 19 nt, Homo sapiens
ucucucugacuccauggca

SEQ ID NO: 5 - RNA, 22 nt, Homo sapiens
uuaucagaaucuccagggguac

SEQ ID NO: 6 - RNA, 22 nt, Homo sapiens
cugcccuagucuagcugaagcu

SEQ ID NO: 7 - RNA, 22 nt, Homo sapiens
uuucuauuucucaguggggcuc

SEQ ID NO: 8 - RNA, 22 nt, Homo sapiens
cacuagauugugagcuccugga

SEQ ID NO: 9 - RNA, 21 nt, Homo sapiens
cuagacugaagcuccuugagg

SEQ ID NO: 10 - RNA, 21 nt, Homo sapiens
cuccguuugccuguuucgcug

SEQ ID NO: 11 - RNA, 22 nt, Homo sapiens
caugccuugaguguaggaccgu

SEQ ID NO: 12 - RNA, 22 nt, Homo sapiens
acugcagugaaggcacuuguag

SEQ ID NO: 13 - RNA, 22 nt, Homo sapiens
cuuucagucggauguuugcagc

SEQ ID NO: 14 - RNA, 98 nt, Homo sapiens
acccaaacccuaggucugcugacuccuaguccagggcucgugauggcuggugggcccugaacgaggggucuggaggccuggguuugaauaucgacagc

SEQ ID NO: 15 - RNA, 110 nt, Homo sapiens
aaccauuagggggcugugguuugccagggcaggagguggaagggagccccauuuacagugguaacuuccuuucccuuuccauccuggcaggcuucagagaacuuuaccag

SEQ ID NO: 16 - RNA, 101 nt, Homo sapiens
gcuggggcuggguggguguggcaggcccaccuuggguaugcaaagcucugacaguguuucacuugcuacccucggucugcuuaccacacucccaguucugc

SEQ ID NO: 17 - RNA, 66 nt, Homo sapiens
aggauucugccauaggaagcuuggaguggaacugaccugcccccuuucucucugacuccauggcag

SEQ ID NO: 18 - RNA, 72 nt, Homo sapiens
ggagcuuaucagaaucuccagggguacuuuauaauuucaaaaagucccccaggugugauucugauuugcuuc

SEQ ID NO: 19 - RNA, 85 nt, Homo sapiens
gggaagggcuucagccaggcuagugcagucugcuuugugccaacacuggggugaugacugcccuagucuagcugaagcuuuuccc

SEQ ID NO: 20 - RNA, 70 nt, Homo sapiens
agugagcaacccagugggcuauggaaauguguggaagauggcauuucuauuucucaguggggcucuuacc

SEQ ID NO: 21 - RNA, 86 nt, Homo sapiens
gguccuugcccucaaggagcucacagucuauugaguuaccuuucugacuuucccacuagauugugagcuccuggagggcaggcacu

SEQ ID NO: 22 - RNA, 90 nt, Homo sapiens
uuuccugcccucgaggagcucacagucuaguaugucucauccccuacuagacugaagcuccuugaggacagggauggucauacucaccuc

SEQ ID NO: 23 - RNA, 86 nt, Homo sapiens
ggugggugguuucuccguuugccuguuucgcugaugugcauucaacucauucucagcaaaauaagcaaauggaaaauucguccauc

SEQ ID NO: 24 - RNA, 91 nt, Homo sapiens
cgacuugcuuucucuccuccaugccuugaguguaggaccguuggcaucuuaauuacccucccacacccaaggcuugcagaagagcgagccu

SEQ ID NO: 25 - RNA, 84 nt, Homo sapiens
gucagaauaaugucaaagugcuuacagugcagguagugauaugugcaucuacugcagugaaggcacuuguagcauuauggugac

SEQ ID NO: 26 - RNA, 71 nt, Homo sapiens
gcgacuguaaacauccucgacuggaagcugugaagccacagaugggcuuucagucggauguuugcagcugc

SEQ ID NO: 27 - RNA, 22 nt, Homo sapiens
cuuucagucggauguuuacagc

SEQ ID NO: 28 - RNA, 22 nt, Homo sapiens
aauguuggaauccucgcuagag

SEQ ID NO: 29 - RNA, 17 nt, Homo sapiens
gcggggcugggcgcgcg

SEQ ID NO: 30 - RNA, 20 nt, Homo sapiens
gugcauugcuguugcauugc

SEQ ID NO: 31 - RNA, 22 nt, Homo sapiens
ccaauauuacugugcugcuuua

SEQ ID NO: 32 - RNA, 23 nt, Homo sapiens
ugagugugugugugugagugugu

SEQ ID NO: 33 - RNA, 21 nt, Homo sapiens
ucagcuacuaccucuauuagg

SEQ ID NO: 34 - RNA, 22 nt, Homo sapiens
aauugcacgguauccaucugua

SEQ ID NO: 35 - RNA, 23 nt, Homo sapiens
ccggucccaggagaaccugcaga

SEQ ID NO: 36 - RNA, 22 nt, Homo sapiens
ugagguaguagguugugugguu

SEQ ID NO: 37 - RNA, 20 nt, Homo sapiens
acuggacuuggagucagaaa

SEQ ID NO: 38 - RNA, 20 nt, Homo sapiens
acugggcuuggagucagaag

SEQ ID NO: 39 - RNA, 23 nt, Homo sapiens
agcuacauugucugcuggguuuc

SEQ ID NO: 40 - RNA, 20 nt, Homo sapiens
acuggacuuggagccagaag

SEQ ID NO: 41 - RNA, 22 nt, Homo sapiens
uccgucucaguuacuuuauagc

SEQ ID NO: 42 - RNA, 22 nt, Homo sapiens
ugagguaguagguuguaugguu

SEQ ID NO: 43 - RNA, 23 nt, Homo sapiens
ccuccguguuaccuguccucuag

SEQ ID NO: 44 - RNA, 22 nt, Homo sapiens
uuauaaagcaaugagacugauu

SEQ ID NO: 45 - RNA, 22 nt, Homo sapiens
uguccucuagggccugcagucu

SEQ ID NO: 46 - RNA, 22 nt, Homo sapiens
cuauacgaccugcugccuuucu

SEQ ID NO: 47 - RNA, 21 nt, Homo sapiens
ucacagugaaccggucucuuu

SEQ ID NO: 48 - RNA, 22 nt, Homo sapiens
ucucugggccugugucuuaggc

SEQ ID NO: 49 - RNA, 22 nt, Homo sapiens
aaaaacugagacuacuuuugca

SEQ ID NO: 50 - RNA, 20 nt, Homo sapiens
aaaaguaauugugguuuuug

SEQ ID NO: 51 - RNA, 22 nt, Homo sapiens
aaaaguaauugugguuuuugcc

SEQ ID NO: 52 - RNA, 20 nt, Homo sapiens
aaaaguaauugugguuuuug

SEQ ID NO: 53 - RNA, 21 nt, Homo sapiens
aaaaguaauugugguuuuugc

SEQ ID NO: 54 - RNA, 23 nt, Homo sapiens
agcagcauuguacagggcuauca

SEQ ID NO: 55 - RNA, 21 nt, Homo sapiens
uaaagugcugacagugcagau

SEQ ID NO: 56 - RNA, 22 nt, Homo sapiens
caaaagugauugugguuuuugc

SEQ ID NO: 57 - RNA, 23 nt, Homo sapiens
agcagcauuguacagggcuauga

SEQ ID NO: 58 - RNA, 22 nt, Homo sapiens
agagguaguagguugcauaguu

SEQ ID NO: 59 - RNA, 22 nt, Homo sapiens
ugauauguuugauauauuaggu

SEQ ID NO: 60 - RNA, 21 nt, Homo sapiens
guuccugcugaacugagccag

SEQ ID NO: 61 - RNA, 22 nt, Homo sapiens
ugucuuacucccucaggcacau

SEQ ID NO: 62 - RNA, 23 nt, Homo sapiens
aaaagugcuuacagugcagguag

SEQ ID NO: 63 - RNA, 22 nt, Homo sapiens
uacgucaucguugucaucguca

SEQ ID NO: 64 - RNA, 22 nt, Homo sapiens
ugugagguuggcauuguugucu

SEQ ID NO: 65 - RNA, 22 nt, Homo sapiens
uuuugugucucccauuccccag

SEQ ID NO: 66 - RNA, 22 nt, Homo sapiens
ugagguaguaguuugugcuguu

SEQ ID NO: 67 - RNA, 23 nt, Homo sapiens
caaagugcuuacagugcagguag

SEQ ID NO: 68 - RNA, 22 nt, Homo sapiens
uacccauugcauaucggaguug

SEQ ID NO: 69 - RNA, 22 nt, Homo sapiens
ucuucaccugccucugccugca

SEQ ID NO: 70 - RNA, 21 nt, Homo sapiens
uacaguacugugauaacugaa

SEQ ID NO: 71 - RNA, 22 nt, Homo sapiens
ugagguaggagguuguauaguu

SEQ ID NO: 72 - RNA, 22 nt, Homo sapiens
uuggcuggucucugcuccgcag

SEQ ID NO: 73 - RNA, 22 nt, Homo sapiens
cuggcccucucugcccuuccgu

SEQ ID NO: 74 - RNA, 21 nt, Homo sapiens
gaucucacuuuguugcccagg

SEQ ID NO: 75 - RNA, 22 nt, Homo sapiens
ugagguaguagauuguauaguu

SEQ ID NO: 76 - RNA, 22 nt, Homo sapiens
ugagguaguagguuguauaguu

SEQ ID NO: 77 - RNA, 23 nt, Homo sapiens
cagugcaauaguauugucaaagc

SEQ ID NO: 78 - RNA, 22 nt, Homo sapiens
uccccuucugcaggccugcugg

SEQ ID NO: 79 - RNA, 23 nt, Homo sapiens
uaaagugcuuauagugcagguag

SEQ ID NO: 80 - RNA, 22 nt, Homo sapiens
uuccugggcuucuccucuguag

SEQ ID NO: 81 - RNA, 21 nt, Homo sapiens
ucucucggcuccucgcggcuc

SEQ ID NO: 82 - RNA, 22 nt, Homo sapiens
ugagguaguaaguuguauuguu

SEQ ID NO: 83 - RNA, 22 nt, Homo sapiens
uucugccucuguccagguccuu

SEQ ID NO: 84 - RNA, 22 nt, Homo sapiens
ugagguaguaguuuguacaguu

(19) United States
(12) Patent Application Publication
(10) Pub. No.: US 2018/0371548 A1
(43) Pub. Date: Dec. 27, 2018

(54) SPECIFIC SIGNATURES IN ALZHEIMER'S DISEASE BY MULTICENTER MICRO-RNA PROFILES

(71) Applicant: SIEMENS SIEMENS AKTIENGESELLSCHAFT, München (DE)

(72) Inventors: Christina Backes, Saarbrücken (DE); Andreas Keller, Saarbrücken (DE); Daniel Sickert, Nürnberg (DE); Cord Friedrich Stähler, Hirschberg an der Bergstraße (DE)

(21) Appl. No.: 16/061,755

(22) PCT Filed: Dec. 14, 2016
(86) PCT No.: PCT/EP2016/081009

§ 371 Date: Jun. 13, 2018

(30) Foreign Application Priority Data
Dec. 22, 2015 (EP) 15201815.6

(51) Int. Cl. C12Q 1/6883

(52) U.S. Cl. CPC C12Q 1/6883; C12Q 2600/158; C12Q 2600/178

(57) ABSTRACT

The disclosure relates to a method for the diagnosis of Alzheimer's disease (AD) in a patient, wherein the expression profile of miRNAs is determined in a blood sample and a comparison of the expression levels of defined miRNAs with a reference sample is carried out. The disclosure also relates to the use of the defined miRNAs as markers for the diagnosis of Alzheimer's disease and to a kit for the diagnosis of Alzheimer's disease.

The present patent document is a § 371 nationalization of PCT Application Serial Number PCT/EP2016/081009, filed Dec. 14, 2016, designating the United States, which is hereby incorporated by reference, and this patent document also claims the benefit of European Patent Application No. EP 15201815.6, filed Dec. 22, 2015, which is also hereby incorporated by reference.

### TECHNICAL FIELD

The disclosure relates to a method for diagnosing Alzheimer's disease (AD) in a patient, in which the expression profile of miRNAs in a blood sample is determined and a comparison of the expression levels of defined miRNAs with a reference sample is carried out, to the use of the defined miRNAs as markers for the diagnosis of Alzheimer's disease, and to a kit for the diagnosis of Alzheimer's disease.

### BACKGROUND

Alzheimer's disease may be diagnosed clinically from the patient's history, from the indirect medical history of relatives and on the basis of clinical observations, based on the presence of characteristic neurological and neuropsychological features and on the absence of alternative states such as other disorders or transient states such as alcoholization (e.g., process of elimination). Moreover, modern medical imaging by computed tomography (CT), magnetic resonance imaging (MRI), single-photon emission computed tomography (SPECT), and/or positron emission tomography (PET) may be used in order to eliminate other cerebral symptoms or subtypes of dementia. It may furthermore predict the transition from prodromal phases (e.g., mild cognitive impairment (MCI)) to Alzheimer's disease. When they are available as a diagnostic tool, SPECT neuroimaging and PET neuroimaging are used in order to confirm an Alzheimer's diagnosis in conjunction with evaluations that include an examination of the mental state. In the case of a person who already has dementia, SPECT appears to be better in the differentiation between Alzheimer's disease and other possible causes in comparison with the usual approaches, which use psychological tests and an analysis of the medical history.

Advances have led to proposals of new diagnostic criteria. A new technique known as PiB PET has been developed in order to be able to directly and clearly depict beta-amyloid deposits in vivo using a tracer which binds selectively to the A-beta deposits. The PiB-PET compound uses carbon-11 PET scanning. More recent studies indicate that PiB-PET is 86% accurate in predicting which patients with a mild cognitive impairment develop Alzheimer's disease within two years, and that this method is accurate to an extent of about 92% in ruling out the likelihood of the development of Alzheimer's disease. It was indicated that amyloid tomography will be used in the future, probably in conjunction with other labels, and not as an alternative. Most recent studies have shown that patients with AD have reduced glutamate levels (Glu) as well as decreased ratios of Glu/creatine (Cr), Glu/myo-inositol (mI), Glu/N-acetylaspartate (NAA), and NAA/Cr in comparison with persons without AD. Both a reduced NAA/Cr ratio and reduced Glu in the hippocampus may be an early indicator of Alzheimer's disease. It was also found out that monitoring deviations in dehydroepiandrosterone (DHEA) in the blood in response to oxidative stress may be a useful test: patients with MCI showed no change in DHEA, whereas the healthy controls showed this. Not long ago, a serum miRNA (microRNA) diagnostic test was proposed (H. Geekiyanage, G. A. Jicha, P. T. Nelson, and C. Chan; Blood serum miRNA: Noninvasive biomarkers for Alzheimer's disease, Exp Neurol. 2012; 235(2), pp. 491-496, doi:10.1016/j.expneurol.2011.11.026).

However, the reliable and early diagnosis of Alzheimer's disease (AD) on the basis of noninvasive molecular biomarkers remains a challenge.

### SUMMARY

The scope of the present disclosure is defined solely by the appended claims and is not affected to any degree by the statements within this summary. The present embodiments may obviate one or more of the drawbacks or limitations in the related art.

To achieve the object of the present disclosure, the creation of an expression profile of a combination of noncoding microRNAs (miRNAs) is proposed for the diagnosis of Alzheimer's disease from blood samples, in particular blood cells. It is possible to diagnose Alzheimer's disease by a change in the expression profile of certain miRNAs in comparison with healthy patients.

According to a first aspect, a method is provided for diagnosing Alzheimer's disease in a patient. The method includes providing a blood sample from a patient; creating an expression profile of miRNAs from the blood sample; and comparing the expression level of at least one miRNA with a reference level, wherein the expression level includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p and/or which includes a sequence deviating from these sequences by, in each case, up to 3 bases, up to 2 bases, or 1 base. The comparison allows for the diagnosis of Alzheimer's disease.

A further aspect provides for the use of a miRNA which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, and/or which includes a sequence deviating from these sequences by, in each case, up to 3 bases, up to 2 bases, or 1 base, as marker for the diagnosis of Alzheimer's disease.

A kit is also provided for the diagnosis of Alzheimer's disease in a blood sample from a patient, including probes and/or primers for the detection of at least one miRNA sequence which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, and/or which includes a sequence deviating from these sequences by, in each case, up to 3 bases, up to 2 bases, or 1 base.

Unless otherwise defined, technical and scientific terms used herein have the same meaning as is generally understood by a person skilled in the art in the field of the disclosure.

### BRIEF DESCRIPTION OF THE DRAWINGS

FIG. 1 depicts an example of a flowchart for diagnosing Alzheimer's disease in a patient.

### DETAILED DESCRIPTION

MicroRNAs or miRNAS or microribonucleic acids are a class of short, noncoding RNAs which, for example, play a role in gene expression. Here, a miRNA is a polynucleotide having a fixed number of bases, for example, of less than 500 bases, less than 200 bases, less than 100 bases, less than 50 bases, or less than 30 bases, but, for example, more than 5 bases, more than 10 bases, or more than 14 bases, and a defined sequence. It is, for example, also possible to obtain cDNA therefrom, and so, in the method, it is also possible to produce from the miRNAs cDNAs which may then likewise be compared with cDNA levels obtained from miRNAs from patients who do not exhibit MS, in particular RRMS.

Hereinafter and above, the references to certain miRNAs are based on the sequences as may be gathered from the Mirbase database (e.g., http://mirbase.org/), in particular Mirbase20, e.g., version 20 of Mirbase.

Furthermore, the sequences which may be used in the method and in the use may also be gathered from Table 1 below. Here, Table 1 specifies the miRNAs in mature form, which may deviate from the hairpin form (e.g., stem loop form).

TABLE 1
Sequences of the miRNAs which may be used in the
method and in the use and in the kit

| | | SEQ ID |
|---|---|---|
| Mature form | Sequence of mature form | NO: |
| hsa-miR-345-5p | gcugacuccuaguccagggcuc | 1 |
| hsa-miR-5006-3p | uuucccuuuccauccuggcag | 2 |
| hsa-miR-7848-3p | cuacccucggucugcuuaccaca | 3 |
| hsa-miR-6817-3p | ucucucugacuccauggca | 4 |
| hsa-miR-361-5p | uuaucagaaucuccagggguac | 5 |
| hsa-miR-3157-3p | cugcccuagucuagcugaagcu | 6 |
| hsa-miR-4482-3p | uuucuauuucucaguggggcuc | 7 |
| hsa-miR-28-3p | cacuagauugugagcuccugga | 8 |
| hsa-miR-151a-3p | cuagacugaagcuccuugagg | 9 |
| hsa-miR-1468-5p | cuccguuugccuguuucgcug | 10 |
| hsa-miR-532-5p | caugccuugaguguaggaccgu | 11 |
| hsa-miR-17-3p | acugcagugaaggcacuuguag | 12 |
| hsa-miR-30a-3p | cuuucagucggauguuugcagc | 13 |

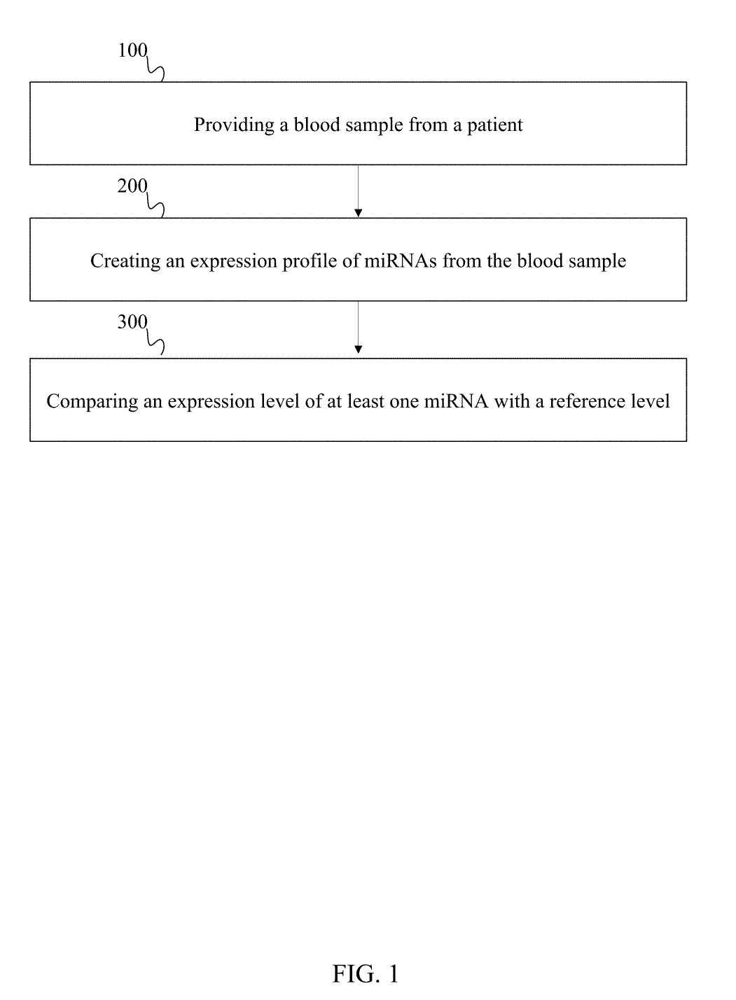

In a first aspect, the present disclosure provides a method for diagnosing Alzheimer's disease in a patient. FIG. 1 depicts an example of one method for diagnosing Alzheimer's disease in a patient. As depicted in FIG. 1, in act 100, a blood sample from a patient is provided. In act 200, an expression profile of miRNAs is created from the blood sample. In act 300, the expression level of at least one miRNA is compared with a reference level, wherein the expression level includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and/or which includes a sequence deviating from these sequences by, in each case, up to 3 bases, up to 2 bases, or 1 base. The comparison allows for the diagnosis of Alzheimer's disease.

Here, the expression profile is a profile of the expression of miRNAs, for example 1, 2, 3, 4, 5, 6, 7, 8, 9, 10, 11, 12, 13, 14, 15, 16, 17, 18, 19, 20, 30, 40, 50, 60, 70, 80, 90, 100, or more miRNAs, in the sample, in quantitative form according to certain embodiments, whereas the expression level indicates the particular quantity of an expressed miRNA.

Here, the provision of the blood sample is not subject to any particular restrictions, but is in particular noninvasive, but makes use of a blood sample that has already been taken. According to certain embodiments, the collection and/or treatment of a blood sample may also be encompassed. Here, a blood sample encompasses a whole blood sample as well as fractions of blood samples such as the plasma, serum, etc., or else cells in the blood sample such as mononuclear cells of the peripheral blood. According to certain embodiments, the blood sample includes blood cells.

The patient from whom the blood sample is provided is likewise not subject to any particular restrictions and may encompass vertebrates, in particular mammals, but is a human according to certain embodiments. In particular, the present method is, according to certain embodiments, used in patients, (e.g., humans), for whom there is a suspicion of Alzheimer's disease and/or for whom there has been a diagnosis of mild cognitive impairment (MCI).

Here, the sequences which are used in the method may be used in a change in the expression level of one or more of the miRNAs which include a corresponding sequence.

The method for creating an expression profile of miRNAs is also not subject to any particular restrictions and may encompass here, for example, common microbiological methods for determining nucleic acids, with use being made here, according to certain embodiments, of a semiquantitative or quantitative method, in particular quantitative method, for the comparison with the reference level. According to certain embodiments, creating the expression profile of miRNAs includes at least one method selected from the group including nucleic acid hybridization, nucleic acid amplification, polymerase extension, sequencing, mass spectrometry, and any combination thereof.

Here, nucleic acid hybridization may encompass the use of arrays, (e.g., microarrays), and/or in situ hybridization. For this purpose, an array may encompass the use of sequences complementary to the certain sequences, (e.g., miRNAs), which include a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and moreover according to certain embodiments additionally miRNAs which include a sequence selected from the group consisting of hsa-miR-28-3p, hsa-miR-151a-3p, hsa-miR-1468-5p, hsa-miR-532-5p, hsa-miR-17-3p, and hsa-miR-30a, and/or which include a sequence deviating from these sequences by, in each case, up to 3 bases, up to 2 bases, or 1 base, which complementary sequences may be appropriately attached to a support. For quantification, it is then possible, for example, to label the miRNAs of the sample with labels such as fluorescent labels or radionucleotide labels.

Alternatively, or additionally, it is also possible to appropriately carry out a polymerase chain reaction (PCR), e.g., a quantitative PCR (qPCR) such as a quantitative real-time PCR (qRT-PCR).

Also conceivable are sequencing methods such as next-generation sequencing and/or mass spectrometry, in particular, quantitative mass spectrometry.

The comparison with a reference level in the method is not subject to any particular restrictions and nay encompass here, in particular, a comparison with the sample from a healthy individual and/or an individual with an MCI diagnosis for whom there is no diagnosis of Alzheimer's disease. Thus, the method allows not only diagnosis, but also staging. By such a comparison, it is, for example, possible to allow the diagnosis of Alzheimer's disease. Here, a comparison may be done on the basis of mathematical approaches such as correlations, on the basis of statistical methods, on the basis of probability theories, on the basis of information-theory approaches, or combinations thereof.

To provide improved information, a statistical comparison using a multiplicity of samples from healthy patients and Alzheimer's patients is advantageous here, wherein such data may also be gathered from a suitable database and/or may be determined on the basis of a mathematical function developed therefrom or an algorithm. Here, the statistical method is not subject to any particular restrictions and may also be carried out in a computer-assisted manner. For example, it is possible to use here a t-test, a Wilcoxon-Mann-Whitney test, determination of area under curve (AUC), etc.

According to certain embodiments, the reference level is obtained from a multiplicity of samples from patients of whom a first group has been diagnosed with AD and a second group exhibits no AD diagnosis, e.g., is healthy in this respect. In certain examples, the groups are adjusting according to age and sex, and with the reference level being determined from the group of healthy patients. To provide more far-reaching information, it is also additionally possible according to certain embodiments to use control groups with a diagnosis of MCI and/or MS. According to certain embodiments, a reference level is determined here from the same kind of sample as the sample to be determined.

Deviating from the sequence is to be understood here to mean a variation in the sequence, for example an addition, an insertion, a deletion or a substitution of a base in the sequence, in comparison with the original sequence, which is specified in Table 1 for example, in particular a deletion and/or an individual base substitution. For example, there may be a deletion of 1 or 2 bases at one end and/or both ends of the sequence, with deletion of no more than 3 bases, no more than 2 bases, 1 base, and in particular no base deletion. Additionally or alternatively, a change of 1 or more bases within the sequence is also possible, there being again maximally a change by 3 bases, 2 bases, 1 base, or no base in comparison with the sequences of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and/or hsa-miR-28-3p, hsa-miR-151a-3p, hsa-miR-1468-5p, hsa-miR-532-5p, hsa-miR-17-3p, and hsa-miR-30a.

According to certain embodiments, there is thus a comparison of the expression level of at least one miRNA with a reference level, where the at least one miRNA includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p).

According to certain embodiments, there is at least one comparison with a sequence including the miRNA hsa-miR-345-5p and/or a sequence deviating from this sequence, e.g., the sequence of the miRNA, by up to 3 bases, up to 2 bases, or 1 base. According to certain embodiments, there is at least one comparison with a sequence including the miRNA hsa-miR-5006-3p and/or a sequence deviating from this sequence by up to 3 bases, up to 2 bases, or 1 base. According to certain embodiments, there is at least one comparison with a sequence including the miRNA hsa-miR-7848-3p and/or a sequence deviating from this sequence by up to 3 bases, up to 2 bases, or 1 base. According to certain embodiments, there is at least one comparison with a sequence including the miRNA hsa-miR-6817-3p and/or a sequence deviating from this sequence by up to 3 bases, up to 2 bases, or 1 base. According to certain embodiments, there is at least one comparison with a sequence including the miRNA hsa-miR-361-5p and/or a sequence deviating from this sequence by up to 3 bases, up to 2 bases, or 1 base. According to certain embodiments, there is at least one comparison with a sequence including the miRNA hsa-miR-3157-3p and/or a sequence deviating from this sequence by up to 3 bases, up to 2 bases, or 1 base. According to certain embodiments, there is at least one comparison with a sequence including the miRNA hsa-miR-4482-3p and/or a sequence deviating from this sequence by up to 3 bases, up to 2 bases, or 1 base.

According to certain embodiments, there is at least one comparison with a sequence including the miRNA hsa-miR-345-5p. According to certain embodiments, there is at least one comparison with a sequence including the miRNA hsa-miR-5006-3p. According to certain embodiments, there is at least one comparison with a sequence including the miRNA hsa-miR-7848-3p. According to certain embodiments, there is at least one comparison with a sequence including the miRNA hsa-miR-6817-3p. According to certain embodiments, there is at least one comparison with a sequence including the miRNA hsa-miR-361-5p. According to certain embodiments, there is at least one comparison with a sequence including the miRNA hsa-miR-3157-3p. According to certain embodiments, there is at least one comparison with a sequence including the miRNA hsa-miR-4482-3p.

According to certain embodiments, the expression level of at least one miRNA is additionally compared with a reference level, wherein the expression level includes a sequence selected from the group consisting of hsa-miR-28-3p, hsa-miR-151a-3p, hsa-miR-1468-5p, hsa-miR-532-5p, hsa-miR-17-3p, and hsa-miR-30a-3p, and/or which includes a sequence deviating from these sequences by, in each case, up to 3 bases, up to 2 bases, or 1 base, wherein the additional comparison allows for the diagnosis of Alzheimer's disease.

According to certain embodiments, a change in the expression level in comparison with a reference level is observed in at least 2, 3, 4, 5, 6, 7, 8, 9, 10, 11, 12, or 13 miRNAs which include a sequence selected from the group consisting of hsa-miR-345-5p, hsa-miR-5006-3p, hsa-miR-7848-3p, hsa-miR-6817-3p, hsa-miR-361-5p, hsa-miR-3157-3p, hsa-miR-4482-3p, hsa-miR-28-3p, hsa-miR-151a-3p, hsa-miR-1468-5p, hsa-miR-532-5p, hsa-miR-17-3p, and hsa-miR-30a-3p, and/or which include a sequence deviating from these sequences by, in each case, up to 3 bases, up to 2 bases, or 1 base. According to certain embodiments, a change in the expression level in comparison with a reference level is observed in at least 2, 3, 4, 5, 6, 7, 8, 9, 10, 11, 12, or 13 miRNAs which include a sequence selected from the group consisting of hsa-miR-345-5p, hsa-miR-5006-3p, hsa-miR-7848-3p, hsa-miR-6817-3p, hsa-miR-361-5p, hsa-miR-3157-3p, hsa-miR-4482-3p, hsa-miR-28-3p, hsa-miR-151a-3p, hsa-miR-1468-5p, hsa-miR-532-5p, hsa-miR-17-3p, and hsa-miR-30a-3p.

According to certain embodiments, a change in the expression level in comparison with a reference level is observed in at least 2, 3, 4, 5, 6, or 7 miRNAs which include a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and/or which include a sequence deviating from these sequences by, in each case, up to 3 bases, up to 2 bases, or 1 base. According to certain embodiments, a change in the expression level in comparison with a reference level is observed in at least 2, 3, 4, 5, 6, or 7 miRNAs which include a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p).

Here, in the case of a change in the expression level of more than one miRNA, the significance level for a diagnosis with Alzheimer's disease may be increased greatly.

According to certain embodiments, the miRNA is at least one selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157), and/or having a sequence deviating from this sequence by up to 3 bases, up to 2 bases, or 1 base. According to certain embodiments, the miRNA is at least one selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157). According to certain embodiments, the further miRNA which is additionally determined in relation to the miRNA which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p and/or which includes a sequence deviating from these sequences by, in each case, up to 3 bases, up to 2 bases, or 1 base, is at least one selected from the group consisting of hsa-miR-28, hsa-miR-151a, hsa-miR-1468, hsa-miR-532, hsa-miR-17, and hsa-miR-30a and/or having a sequence deviating from this sequence by up to 3 bases, up to 2 bases, or 1 base. According to certain embodiments, the further miRNA which is additionally determined in relation to the miRNA which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, and/or which includes a sequence deviating from these sequences by, in each case, up to 3 bases, up to 2 bases, or 1 base, is at least one selected from the group consisting of hsa-miR-28, hsa-miR-151a, hsa-miR-1468, hsa-miR-532, hsa-miR-17, and hsa-miR-30a. Thus, according to certain embodiments, the expression level of at least one miRNA selected from the group consisting of hsa-miR-28, hsa-miR-151a, hsa-miR-1468, hsa-miR-532, hsa-miR-17, and hsa-miR-30a is additionally determined.

Here, these embodiments of the miRNA correspond to the stem loop form or hairpin form, which correlates with the detected mature form as shown in Table 2 (e.g., both for the miRNAs of the use and for the further miRNAs usable in the method) and which may be present as such in the samples. Accordingly, instead of with the particular mature form, the comparison may also be done with the corresponding stem loop form.

TABLE 2
Correlation between stem loop form and mature form

| Stem loop form | SEQ ID NO: | Mature form | Sequence of mature form |
|---|---|---|---|
| hsa-miR-345 | 14 | hsa-miR-345-5p | gcugacuccuaguccagggcuc |
| hsa-miR-5006 | 15 | hsa-miR-5006-3p | uuucccuuuccauccuggcag |
| hsa-miR-7848 | 16 | hsa-miR-7848-3p | cuacccucggucugcuuaccaca |
| hsa-miR-6817 | 17 | hsa-miR-6817-3p | ucucucugacuccauggca |
| hsa-miR-361 | 18 | hsa-miR-361-5p | uuaucagaaucuccagggguac |
| hsa-miR-3157 | 19 | hsa-miR-3157-3 | pcugcccuagucuagcugaagcu |
| hsa-miR-4482 | 20 | hsa-miR-4482-3p | uuucuauuucucaguggggcuc |
| hsa-miR-28 | 21 | hsa-miR-28-3p | cacuagauugugagcuccugga |
| hsa-miR-151a | 22 | hsa-miR-151a-3 | pcuagacugaagcuccuugagg |
| hsa-miR-1468 | 23 | hsa-miR-1468-5p | cuccguuugccuguuucgcug |
| hsa-miR-532 | 24 | hsa-miR-532-5p | caugccuugaguguaggaccgu |
| hsa-miR-17 | 25 | hsa-miR-17-3p | acugcagugaaggcacuuguag |
| hsa-miR-30a | 26 | hsa-miR-30a-3p | cuuucagucggauguuugcagc |

The nucleic acid sequences of the stem loop forms according to Table 2 are as follows:

14:
acccaaaccc uaggucugcu gacuccuagu ccagggcucg
ugauggcugg ugggcccuga acgagggguc uggaggccug
gguuugaaua ucgacagc
15:
aaccauuagg gggcuguggu uugccagggc aggaggugga
agggagcccc auuuacagug guaacuuccu uucccuuucc
auccuggcag gcuucagaga acuuuaccag
16:
gcuggggcug ggugggugug gcaggcccac cuuggguaug
caaagcucug acaguguuuc acuugcuacc cucggucugc
uuaccacacu cccaguucug c
17:
aggauucugc cauaggaagc uuggagugga acugaccugc
ccccuuucuc ucugacucca uggcag
18:
ggagcuuauc agaaucucca gggguacuuu auaauuucaa
aaaguccccc aggugugauu cugauuugcu uc
19:
gggaagggcu ucagccaggc uagugcaguc ugcuuugugc
caacacuggg gugaugacug cccuagucua gcugaagcuu
uuccc
20:
agugagcaac ccagugggcu auggaaaugu guggaagaug
gcauuucuau uucucagugg ggcucuuacc
21:
gguccuugcc cucaaggagc ucacagucua uugaguuacc
uuucugacuu ucccacuaga uugugagcuc cuggagggca
ggcacu
22:
uuuccugccc ucgaggagcu cacagucuag uaugucucau
ccccuacuag acugaagcuc cuugaggaca gggaugguca
uacucaccuc
23:
gguggguggu uucuccguuu gccuguuucg cugaugugca
uucaacucau ucucagcaaa auaagcaaau ggaaaauucg
uccauc
24:
cgacuugcuu ucucuccucc augccuugag uguaggaccg
uuggcaucuu aauuacccuc ccacacccaa ggcuugcaga
agagcgagcc u
25:
gucagaauaa ugucaaagug cuuacagugc agguagugau
augugcaucu acugcaguga aggcacuugu agcauuaugg
ugac
26:
gcgacuguaa acauccucga cuggaagcug ugaagccaca
gaugggcuuu cagucggaug uuugcagcug c

According to certain embodiments, a change in the expression level in comparison with a reference level is observed in at least 2, 3, 4, 5, 6, or 7 miRNAs which include a sequence selected from the group consisting of hsa-miR-345, hsa-miR-5006, hsa-miR-7848, hsa-miR-6817, hsa-miR-361, hsa-miR-3157, and hsa-miR-4482 and/or which include a sequence deviating from these sequences by, in each case, up to 3 bases, up to 2 bases, or 1 base. According to certain embodiments, a change in the expression level in comparison with a reference level is observed in at least 2, 3, 4, 5, 6, or 7 miRNAs which include a sequence selected from the group consisting of hsa-miR-345, hsa-miR-5006, hsa-miR-7848, hsa-miR-6817, hsa-miR-361, hsa-miR-3157, and hsa-miR-4482.

According to a further aspect, the disclosure provides for the use of at least one miRNA which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and/or which includes a sequence deviating from these sequences by, in each case, up to 3 bases, up to 2 bases, or 1 base, as marker for the diagnosis of Alzheimer's disease. According to certain embodiments, at least one miRNA which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), is used.

Here, according to certain embodiments, the miRNA is at least one which has a sequence selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157), and/or which has a sequence deviating from these sequences by, in each case, up to 3 bases, up to 2 bases, or 1 base. Here, according to certain embodiments, the miRNA is at least one which has a sequence selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157).

In a further aspect, the present disclosure provides a kit for the diagnosis of Alzheimer's disease in a blood sample from a patient, including probes and/or primers for the detection of at least one miRNA sequence which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p), and/or which includes a sequence deviating from these sequences by, in each case, up to 3 bases, up to 2 bases, or 1 base.

According to certain embodiments, the kit includes probes and/or primers for the detection of at least one miRNA sequence which includes a sequence selected from the group consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, (e.g., hsa-miR-345-5p, hsa-miR-5006-3p, hsa-miR-361-5p, or hsa-miR-3157-3p).

According to certain embodiments, the kit includes probes and/or primers for the detection of at least one miRNA sequence which has a sequence selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157), and/or which has a sequence deviating from these sequences by, in each case, up to 3 bases, up to 2 bases, or 1 base.

According to certain embodiments, the kit includes probes and/or primers for the detection of at least one miRNA sequence which has a sequence selected from the group consisting of hsa-miR-345, hsa-miR-5006, hsa-miR-7848, hsa-miR-6817, hsa-miR-361, hsa-miR-3157, and hsa-miR-4482, (e.g., hsa-miR-345, hsa-miR-5006, hsa-miR-361, or hsa-miR-3157).

The kit may additionally include probes and/or primers for the detection of at least one miRNA sequence which includes a sequence selected from the group consisting of hsa-miR-28-3p, hsa-miR-151a-3p, hsa-miR-1468-5p, hsa-miR-532-5p, hsa-miR-17-3p, and hsa-miR-30a-3p and/or which includes a sequence deviating from these sequences by, in each case, up to 3 bases, up to 2 bases, or 1 base. In one example, the kit may include probes and/or primers for the detection of at least one miRNA sequence including a sequence selected from the group consisting of hsa-miR-28-3p, hsa-miR-151a-3p, hsa-miR-1468-5p, hsa-miR-532-5p, hsa-miR-17-3p, and hsa-miR-30a-3p. The kit may additionally include probes and/or primers for the detection of at least one miRNA sequence which has a sequence selected from the group consisting of hsa-miR-28, hsa-miR-151a, hsa-miR-1468, hsa-miR-532, hsa-miR-17, and hsa-miR-30a, and/or which has a sequence deviating from these sequences by, in each case, up to 3 bases, up to 2 bases, or 1 base. In example, the kit may include probes and/or primers for the detection of at least one miRNA sequence which has a sequence selected from the group consisting of hsa-miR-28, hsa-miR-151a, hsa-miR-1468, hsa-miR-532, hsa-miR-17, and hsa-miR-30a.

According to certain embodiments, the kit includes probes and/or primers for the detection of 2, 3, 4, 5, 6, 7, 8, 9, 10, 11, 12, or 13 of the above sequences.

Here, the probes and/or primers are not subject to any particular restrictions and may include oligomers which have a nucleotide sequence, for example a cDNA sequence as well, which is complementary to the sequence to be detected.

In addition, it is also possible to provide further probes and/or primers as controls, for example of further miRNAs for which there is no change in the expression profile in comparison with patients without AD. It is also possible to provide controls for nonspecific binding and/or hybridization.

According to certain embodiments, the primers and/or probes and, optionally, controls may be applied to a suitable substrate.

According to certain embodiments, the kit further includes enzymes and/or reagents for carrying out an RT-PCR, in particular qRT-PCR, these not being subject to any particular restrictions. Here, reagents may also encompass labels, for example for fluorescence labeling and/or radionucleotide labeling. The kit may also include reagents for cDNA synthesis from the miRNAs before the PCR, e.g., qPCR and/or RT-PCR, in particular qRT-PCR.

The above embodiments, configurations and developments may, where meaningful, be combined with one another in any manner. Further possible configurations, developments and implementations of the disclosure also encompass nonexplicitly stated combinations of features of the disclosure, which features are described above or are described below with regard to the exemplary embodiments. In particular, a person skilled in the art will also add individual aspects as improvements or supplements to the particular basic form of the present disclosure.

The disclosure will be further elucidated below on the basis of exemplary embodiments, which, however, do not restrict the disclosure.

### Example 1

In the example, samples from individuals were examined by high-throughput sequencing as unbiased technology. A blind multicentric case-control study was carried out with 294 individuals, including 107 AD patients, 20 MCI patients, 90 MS patients, and 77 age- and sex-adjusted controls. The miRNomes (e.g., entireties of the microRNAs) of all the individuals were examined using next-generation sequencing (NGS) in the usual manner. Here, the AD patients and controls were split into a first cohort with 54 AD patients and 22 controls from the United States, who were tested first, and a second replication cohort with 53 AD patients and 55 controls from a German university hospital.

When carrying out the NGS with the 294 individuals, approximately 6 billion short RNA sequences were generated as reads. An AD group and a control group were profiled from the data from the USA and Germany. Altogether 586 miRNAs were found in the blood. In the US cohort, 210 miRNAs were changed in expression between the AD and control samples, and in the German cohort, 135 miRNAs were changed. In both studies, a significant (p<0.00001) overlap of 69 miRNAs was found. Of these, the expression direction of 96% agreed in both cohorts, and the area under curve (AUC) values showed a highly significant correlation (p<10⁻¹⁶) of 0.92 (95% confidence interval (CI) of 0.87-0.95).

The statistically significant miRNAs found herein, which may be used for the diagnosis of Alzheimer's, are given in Table 3.

The miRNAs in the signature appear to be involved in mitochondrial dysfunction via the potential target genes PARL (p=0.004) and SLC25A5 (p=0.002).

TABLE 3
miRNAs with significantly different expression level in AD patients in
comparison with the control group

| Mature form | t-test, AD vs. control | AUC, AD vs. control |
|---|---|---|
| hsa-miR-345-5p | 3.20E−04 | 0.33 |
| hsa-miR-5006-3p | 1.26E−04 | 0.36 |
| hsa-miR-7848-3p | 2.28E−03 | 0.40 |
| hsa-miR-6817-3p | 1.02E−03 | 0.42 |
| hsa-miR-361-5p | 1.25E−07 | 0.29 |
| hsa-miR-3157-3p | 1.14E−07 | 0.29 |
| hsa-miR-4482-3p | 3.46E−03 | 0.35 |
| hsa-miR-28-3p | 1.70E−10 | 0.24 |
| hsa-miR-151a-3p | 7.13E−11 | 0.25 |
| hsa-miR-1468-5p | 7.11E−08 | 0.32 |
| hsa-miR-532-5p | 6.94E−04 | 0.65 |
| hsa-miR-17-3p | 5.63E−08 | 0.78 |
| hsa-miR-30a-3p | 1.65E−08 | 0.24 |

Further relevant mature miRNA sequences found for the two examined cohorts, which were determined on the basis of a Wilcoxon-Mann-Whitney test, are shown in Table 4 with the values found from, in each case, one Wilcoxon-Mann-Whitney test carried out per cohort.

TABLE 4
miRNAs with different expression level in AD patients in comparison with
the control group on the basis of the results of Wilcoxon-Mann-Whitney tests
for the first and second cohort

| | | | Wilcoxon- | Wilcoxon- |
|---|---|---|---|---|
| | | | Mann- | Mann- |
| | | SEQ | Whitney | Whitney |
| | | ID | test | test |
| miRNA | Mature sequence | NO: | (1st cohort) | (2nd cohort) |
| hsa-miR-17-3p | acugcagugaaggcacuuguag | 12 | 1.15E-05 | 0.00020271 |
| hsa-miR-30a-3p | cuuucagucggauguuugcagc | 13 | 0.0035552 | 4.14E-06 |
| hsa-miR-28-3p | cacuagauugugagcuccugga | 8 | 0.00038033 | 6.33E-06 |
| hsa-miR-30e-3p | cuuucagucggauguuuacagc | 27 | 0.0073513 | 2.34E-06 |
| hsa-miR-151a-3p | cuagacugaagcuccuugagg | 9 | 1.70E-07 | 0.0045649 |
| hsa-miR-4781-3p | aauguuggaauccucgcuagag | 28 | 2.44E-06 | 0.043509 |
| hsa-miR-4508 | gcggggcugggcgcgcg | 29 | 0.020063 | 0.0011682 |
| hsa-miR-361-5p | uuaucagaaucuccagggguac | 5 | 3.13E-05 | 0.00010372 |
| hsa-miR-3157-3p | cugcccuagucuagcugaagcu | 6 | 5.74E-06 | 0.0052611 |
| hsa-miR-33b-5p | gugcauugcuguugcauugc | 30 | 9.95E-05 | 0.0035992 |
| hsa-miR-16-2-3p | ccaauauuacugugcugcuuua | 31 | 0.027024 | 0.00018878 |
| hsa-miR-574-5p | ugagugugugugugugagugugu | 32 | 0.025881 | 0.0046907 |
| hsa-miR-5690 | ucagcuacuaccucuauuagg | 33 | 0.0023133 | 0.0067456 |
| hsa-miR-363-3p | aauugcacgguauccaucugua | 34 | 0.0043552 | 0.0017395 |
| hsa-miR-4746-5p | ccggucccaggagaaccugcaga | 35 | 0.018015 | 0.003912 |
| hsa-let-7b-5p | ugagguaguagguugugugguu | 36 | 0.029466 | 0.0060788 |
| hsa-miR-1468-5p | cuccguuugccuguuucgcug | 10 | 0.0041147 | 0.0019343 |
| hsa-miR-345-5p | gcugacuccuaguccagggcuc | 1 | 0.0012034 | 0.019022 |
| hsa-miR-378d | acuggacuuggagucagaaa | 37 | 0.0027945 | 0.00033694 |
| hsa-miR-378g | acugggcuuggagucagaag | 38 | 0.0031832 | 0.00021093 |
| hsa-miR-221-3p | agcuacauugucugcuggguuuc | 39 | 0.004188 | 0.0068757 |
| hsa-miR-378f | acuggacuuggagccagaag | 40 | 0.0024916 | 0.0023304 |
| hsa-miR-340-3p | uccgucucaguuacuuuauagc | 41 | 0.0002208 | 0.00063591 |
| hsa-let-7c-5p | ugagguaguagguuguaugguu | 42 | 8.00E-06 | 0.0047904 |
| hsa-miR-532-5p | caugccuugaguguaggaccgu | 11 | 0.0056523 | 0.0061427 |
| hs a-miR-3605-3p | ccuccguguuaccuguccucuag | 43 | 0.031272 | 8.42E-06 |
| hsa-miR-340-5p | uuauaaagcaaugagacugauu | 44 | 0.00092804 | 0.0023566 |
| hsa-miR-5006-3p | uuucccuuuccauccuggcag | 2 | 0.015025 | 0.034036 |
| hsa-miR-3909 | uguccucuagggccugcagucu | 45 | 0.00072755 | 0.0072214 |
| hsa-let-7d-3p | cuauacgaccugcugccuuucu | 46 | 0.014922 | 0.00060374 |
| hsa-miR-128-3p | ucacagugaaccggucucuuu | 47 | 1.04E-05 | 0.010414 |
| hsa-miR-330-5p | ucucugggccugugucuuaggc | 48 | 7.40E-05 | 0.00086506 |
| hsa-miR-548e-3p | aaaaacugagacuacuuuugca | 49 | 0.0090437 | 0.036586 |
| hsa-miR-548ad-5p | aaaaguaauugugguuuuug | 50 | 0.0047025 | 0.00040004 |
| hsa-miR-548d-5p | aaaaguaauugugguuuuugcc | 51 | 0.0047025 | 0.00040004 |
| hsa-miR-548ae-5p | aaaaguaauugugguuuuug | 52 | 0.0031776 | 0.0040292 |
| hsa-miR-548ay-5p | aaaaguaauugugguuuuugc | 53 | 0.0031776 | 0.0040292 |
| hsa-miR-107 | agcagcauuguacagggcuauca | 54 | 6.13E-05 | 0.00026867 |
| hsa-miR-106b-5p | uaaagugcugacagugcagau | 55 | 0.028274 | 0.0020005 |
| hsa-miR-548az-5p | caaaagugauugugguuuuugc | 56 | 0.0051462 | 0.0037591 |
| hsa-miR-103 a-3p | agcagcauuguacagggcuauga | 57 | 0.00017315 | 0.00048883 |
| hsa-let-7d-5p | agagguaguagguugcauaguu | 58 | 0.014163 | 0.0079347 |
| hsa-miR-190a-5p | ugauauguuugauauauuaggu | 59 | 1.61E-05 | 0.0019051 |
| hsa-miR-3074-5p | guuccugcugaacugagccag | 60 | 0.0081401 | 0.018552 |
| hsa-miR-550a-3p | ugucuuacucccucaggcacau | 61 | 0.0066173 | 0.0034703 |
| hsa-miR-106a-5p | aaaagugcuuacagugcagguag | 62 | 0.014462 | 0.016999 |
| hsa-miR-598-3p | uacgucaucguugucaucguca | 63 | 0.0042481 | 0.0055876 |
| hsa-miR-1294 | ugugagguuggcauuguugucu | 64 | 1.88E-05 | 0.026805 |
| hsa-miR-5010-3p | uuuugugucucccauuccccag | 65 | 2.25E-05 | 0.037426 |
| hsa-let-7i-5p | ugagguaguaguuugugcuguu | 66 | 0.00012721 | 0.042682 |
| hsa-miR-17-5p | caaagugcuuacagugcagguag | 67 | 0.039237 | 0.033477 |
| hsa-miR-660-5p | uacccauugcauaucggaguug | 68 | 3.50E-06 | 0.011728 |
| hsa-miR-6754-3p | ucuucaccugccucugccugca | 69 | 0.018408 | 0.013335 |
| hsa-miR-101-3p | uacaguacugugauaacugaa | 70 | 1.06E-06 | 0.0019576 |
| hsa-let-7e-5p | ugagguaggagguuguauaguu | 71 | 8.31E-07 | 0.0034125 |
| hsa-miR-6842-3p | uuggcuggucucugcuccgcag | 72 | 0.0030116 | 0.0022308 |
| hsa-miR-328-3p | cuggcccucucugcccuuccgu | 73 | 0.0087011 | 0.0018858 |
| hsa-miR-1285-5p | gaucucacuuuguugcccagg | 74 | 6.09E-05 | 0.0048518 |
| hsa-let-7f-5p | ugagguaguagauuguauaguu | 75 | 4.82E-09 | 0.0028229 |
| hsa-let-7a-5p | ugagguaguagguuguauaguu | 76 | 1.51E-08 | 0.004414 |
| hsa-miR-301a-3p | cagugcaauaguauugucaaagc | 77 | 0.0018718 | 0.012808 |
| hsa-miR-3127-3p | uccccuucugcaggccugcugg | 78 | 2.04E-05 | 0.045572 |
| hsa-miR-20a-5p | uaaagugcuuauagugcagguag | 79 | 0.025138 | 0.010058 |
| hsa-miR-6783-3p | uuccugggcuucuccucuguag | 80 | 0.029479 | 0.0093665 |
| hsa-miR-3615 | ucucucggcuccucgcggcuc | 81 | 0.016317 | 0.046337 |
| hsa-miR-98-5p | ugagguaguaaguuguauuguu | 82 | 9.18E-07 | 0.020253 |
| hsa-miR-5001-3p | uucugccucuguccagguccuu | 83 | 2.24E-06 | 0.01183 |
| hsa-let-7g-5p | ugagguaguaguuuguacaguu | 84 | 1.81E-07 | 0.017748 |

### Example 2

The specificity of the miRNAs found in Example 1 for Alzheimer's disease was assessed by additionally comparing the expression level thereof with those of MCI and MS (multiple sclerosis) patients, which were obtained as in Example 1. The results in relation thereto are shown in Table 5.

TABLE 5
miRNAs with significantly different expression level in AD patients in
comparison with MCI and MS patients

| | t-test, | | t-test, | AUC, |
|---|---|---|---|---|
| | AD vs. | AUC, AD vs. | AD vs. | AD vs. |
| Mature form | MCI | MCI | MS | MS |
| hsa-miR-345-5p | 4.96E−02 | 0.38 | 6.83E−04 | 0.39 |
| hsa-miR-5006-3p | 2.88E−02 | 0.32 | 1.16E−02 | 0.37 |
| hsa-miR-7848-3p | 8.76E−01 | 0.44 | 1.81E−06 | 0.27 |
| hsa-miR-6817-3p | 5.03E−01 | 0.40 | 3.98E−10 | 0.28 |
| hsa-miR-361-5p | 3.81E−02 | 0.28 | 3.93E−04 | 0.36 |
| hsa-miR-3157-3p | 1.74E−02 | 0.29 | 5.17E−13 | 0.21 |
| hsa-miR-4482-3p | 8.30E−02 | 0.38 | 4.83E−03 | 0.37 |
| hsa-miR-28-3p | 3.56E−01 | 0.44 | 1.24E−06 | 0.32 |
| hsa-miR-151a-3p | 5.87E−01 | 0.44 | 1.29E−15 | 0.20 |
| hsa-miR-1468-5p | 7.11E−02 | 0.39 | 8.61E−17 | 0.14 |
| hsa-miR-532-5p | 5.89E−03 | 0.71 | 4.37E−21 | 0.87 |
| hsa-miR-17-3p | 2.59E−01 | 0.59 | 1.23E−16 | 0.82 |
| hsa-miR-30a-3p | 5.84E−01 | 0.58 | 6.06E−03 | 0.44 |

Whereas the most significant AD miRNAs from the panel from Example 1 were also dysregulated between MS and AD, MCI patients were more similar to AD patients.

Because the same panel of miRNAs may also distinguish between MCI, MS, and AD, it may be inferred that the miRNAs in the signature found are specific for Alzheimer's disease and may be accordingly used for an early detection or diagnosis of the disease.

Although the disclosure has been illustrated and described in detail by the exemplary embodiments, the disclosure is not restricted by the disclosed examples and the person skilled in the art may derive other variations from this without departing from the scope of protection of the disclosure. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

1. A method for diagnosing Alzheimer's disease in a patient, the method comprising:
providing a blood sample from the patient;
creating an expression profile of miRNAs from the blood sample; and
comparing an expression level of at least one miRNA with a reference level, wherein the expression level comprises one or both of:
a sequence selected from a group of sequences consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, or
a sequence deviating from the group of sequences by, in each case, up to 3 bases,
wherein the comparison allows for a diagnosis of Alzheimer's disease.

2. The method of claim 1, wherein the expression level of at least one additional miRNA comprises one or both of:
a sequence selected from an additional group of sequences consisting of hsa-miR-28-3p, hsa-miR-151a-3p, hsa-miR-1468-5p, hsa-miR-532-5p, hsa-miR-17-3p and hsa-miR-30a-3p, or
a sequence deviating from the additional group of sequences by, in each case, up to 3 bases,
wherein the at least one additional miRNA is additionally compared with a reference level,
wherein the additional comparison allows for the diagnosis of Alzheimer's disease.

3. The method of claim 1, wherein the patient is a human.

4. The method of claim 1, wherein a change in the expression level in comparison with a reference level is observed in at least 2, 3, 4, 5, 6, 7, 8, 9, 10, 11, 12, or 13 miRNAs.

5. The method of claim 1, wherein the expression level of the at least one miRNA is selected from the group consisting of hsa-miR-345, hsa-miR-5006, hsa-miR-7848, hsa-miR-6817, hsa-miR-361, hsa-miR-3157, hsa-miR-4482, and combinations thereof.

6. The method of claim 2, wherein the expression level of at least one additional miRNA is selected from the group consisting of hsa-miR-28, hsa-miR-151a, hsa-miR-1468, hsa-miR-532, hsa-miR-17, hsa-miR-30a, and combinations thereof.

7. The method of claim 1, wherein the creating of the expression profile comprises nucleic acid hybridization, nucleic acid amplification, polymerase extension, sequencing, mass spectrometry, or any combination thereof.

8.-9. (canceled)

10. A kit for the diagnosis of Alzheimer's disease in a blood sample from a patient, the kit comprising:
probes, primers, or both the probes and the primers configured to detect at least one miRNA sequence comprises one or both of:
a sequence selected from a group of sequences consisting of hsa-miR-345-5p, hsa-miR-5006-3p, hsa-miR-7848-3p, hsa-miR-6817-3p, hsa-miR-361-5p, hsa-miR-3157-3p, and hsa-miR-4482-3p, or
a sequence deviating from the group of sequences sequences by, in each case, up to 3 bases.

11. The kit of claim 10, further comprising;
additional probes, additional primers, or both the additional probes and the additional primers configured to detect at least one additional miRNA sequence comprises one or both of:
a sequence selected from an additional group of sequences consisting of hsa-miR-28-3p, hsa-miR-151a-3p, hsa-miR-1468-5p, hsa-miR-532-5p, hsa-miR-17-3p, and hsa-miR-30a-3p, or
a sequence deviating from the additional group of sequences by, in each case, up to 3 bases.

12. The kit of claim 10, further comprising:
enzymes, reagents, or both the enzymes and the reagents configured to carry out a real-time polymerase chain reaction.

13. The kit of claim 10, wherein the sequence deviates from the group of sequences by up to 2 bases.

14. The kit of claim 10, wherein the sequence deviates from the group of sequences by 1 base.

15. The kit of claim 11, wherein the sequence deviates from the additional group of sequences by up to 2 bases.

16. The kit of claim 11, wherein the sequence deviates from the additional group of sequences by 1 base.

17. The method of claim 1, wherein the sequence deviates from the group of sequences by up to 2 bases.

18. The method of claim 1, wherein the sequence deviates from the group of sequences by 1 base.

19. The method of claim 2, wherein the sequence deviates from the additional group of sequences by up to 2 bases.

20. The method of claim 2, wherein the sequence deviates from the additional group of sequences by 1 base.
